(19) United States
(12) Patent Application Publication
(10) Pub. No.: US 2026/0285630 A1
(43) Pub. Date: Sep. 24, 2026

(54) AUTOMATIC DOCUMENT FEEDER AND AUTOMATIC DOCUMENT FEEDING METHOD

(71) Applicant: Hirofumi Ono, Kanagawa (JP)

(72) Inventor: Hirofumi Ono, Kanagawa (JP)

(73) Assignee: ETRIA Co., Ltd., Kanagawa (JP)

(21) Appl. No.: 19/561,986

(22) Filed: Mar. 10, 2026

(30) Foreign Application Priority Data
Mar. 19, 2025 (JP) 2025-044574

(51) Int. Cl. B65H 7/14; B65H 1/04; B65H 3/06; B65H 9/04; G01N 21/3563; G01N 33/34

(52) U.S. Cl. CPC B65H 7/14; B65H 1/04; B65H 3/0684; B65H 9/04; G01N 21/3563; G01N 33/346; B65H 2301/423245; B65H 2551/18; B65H 2553/414; B65H 2557/64

(57) ABSTRACT

An automatic document feeder includes a document stacker to stack a document; a sheet feeder; a conveyor; a detector; and circuitry. The sheet feeder feeds the document from the document stacker includes a pickup roller; and a pressure member to apply a pressure to press the pickup roller against the document on the document stacker. The conveyor conveys the document fed by the sheet feeder in a conveyance direction. The detector detects document information including at least a type of the document on the document stacker. The circuitry is configured to determine whether the type of the document on the document stacker is a thermally-developable document based on the document information detected by the detector, and control the pressure member to change the pressure applied to the pickup roller based on a determination of the type of the document on the document stacker.

### CROSS-REFERENCE TO RELATED APPLICATIONS

This patent application is based on and claims priority pursuant to 35 U.S.C. § 119 (a) to Japanese Patent Application No. 2025-044574, filed on Mar. 19, 2025, in the Japan Patent Office, the entire disclosure of which is hereby incorporated by reference herein.

### BACKGROUND

### Technical Field

The present disclosure relates to an automatic document feeder and an automatic document feeding method.

### Related Art

In order to prevent a document sheet closed at one end from being torn or jammed, an automatic document feeder has been proposed in which, when a user selects a document sheet mode, a pressure applied by a pickup roller to a document sheet is increased to separate a separation roller from a feed roller.

Such a technique changes the pressure of the pickup roller depending on a document sheet type. However, in such techniques, when a document that develops color by heat, such as thermal paper, is fed, contamination may occur on the document in a sheet feeding section.

### SUMMARY

An embodiment of the present disclosure provides an automatic document feeder includes a document stacker to stack a document; a sheet feeder; a conveyor; a detector; and circuitry. The sheet feeder feeds the document from the document stacker includes a pickup roller; and a pressure member to apply a pressure to press the pickup roller against the document on the document stacker. The conveyor conveys the document fed by the sheet feeder in a conveyance direction. The detector detects document information including at least a type of the document on the document stacker. The circuitry is configured to determine whether the type of the document on the document stacker is a thermally-developable document based on the document information detected by the detector; and control the pressure member to change the pressure applied to the pickup roller based on a determination of the type of the document on the document stacker.

### BRIEF DESCRIPTION OF THE DRAWINGS

A more complete appreciation of embodiments of the present disclosure and many of the attendant advantages and features thereof can be readily obtained and understood from the following detailed description with reference to the accompanying drawings, wherein:

FIG. 1 is a diagram illustrating an area in and around a sheet feeder of an automatic document feeder in a typical configuration, according to a comparative example;

FIG. 2 is a diagram illustrating a thermal paper document sheet fed by the automatic document feeder in FIG. 1;

FIG. 3 is a diagram illustrating an automatic document feeder according to a first embodiment;

FIG. 4 is a block diagram of a sheet feeder in FIG. 1;

FIG. 5 is a diagram illustrating a configuration of a pressure member including a driving device;

FIG. 6 is a plan view of the automatic document feeder in FIG. 3 as viewed from above;

FIG. 7 is a block diagram of a sheet feeding section according to a second embodiment;

FIG. 8 is a diagram illustrating an automatic document feeder according to a third embodiment;

FIG. 9 is a diagram illustrating an automatic document feeder according to a fourth embodiment; and

FIG. 10 is a block diagram of a hardware configuration of a controller.

The accompanying drawings are intended to depict embodiments of the present disclosure and should not be interpreted to limit the scope thereof. The accompanying drawings are not to be considered as drawn to scale unless explicitly noted. Also, identical or similar reference numerals designate identical or similar components throughout the several views.

### DETAILED DESCRIPTION

In describing embodiments illustrated in the drawings, specific terminology is employed for the sake of clarity. However, the disclosure of this specification is not intended to be limited to the specific terminology so selected and it is to be understood that each specific element includes all technical equivalents that have a similar function, operate in a similar manner, and achieve a similar result.

Referring now to the drawings, embodiments of the present disclosure are described below. As used herein, the singular forms “a,” “an,” and “the” are intended to include the plural forms as well, unless the context clearly indicates otherwise.

According to one aspect of the present disclosure, when a document that develops color upon heating, such as thermal paper, is fed, contamination of the document in a sheet feeder can be prevented.

Embodiments of the present disclosure are described below with reference to the accompanying drawings. In order to facilitate the understanding of the description, like reference signs denote like elements in the drawings, and overlapping description may be simplified or omitted as appropriate.

### Challenges in a Typical Configuration (Comparative Example)

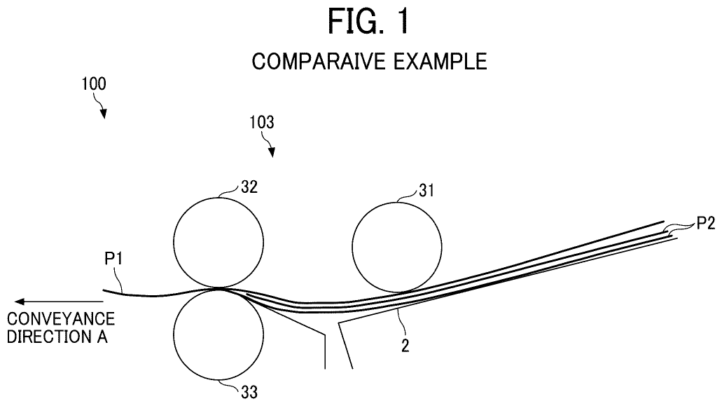

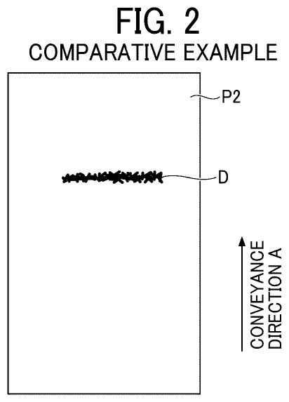

Challenges in a typical configuration are described with reference to FIGS. 1 and 2.

FIG. 1 is a diagram illustrating an area in and around a sheet feeder 103 of an automatic document feeder 100 in a typical configuration, according to a comparative example. With reference to FIG. 1, the contamination mechanism of the thermal paper and the challenges in a typical configuration are described.

As illustrated in FIG. 1, the sheet feeder 103 includes a pickup roller 31, a feed roller 32 and a reverse roller 33. The pickup roller 31 feeds a document P1 from a document stacker 2 in the conveyance direction A. The feed roller 32 and the reverse roller 33 are disposed downstream of the pickup roller 31 in a conveyance direction A and are arranged to face each other with the document P1 interposed therebetween.

When the pickup roller 31 feeds the document P1, such as thermal paper that develops color upon heating, stacked on the document stacker 2, a back surface of the document P1 and a front surface of a stacked document P2 (a next document) are rubbed against each other at a pickup portion (a portion pressed by the pickup roller 31). As a result, frictional heat causes the front surface of the stacked document P2 to undergo a thermosensitive reaction, thus forming a stain D (see FIG. 2).

Since the frictional force increases as the pressure with which the pickup roller 31 presses the document P1 increases, the stain D formed on the stacked document P2 due to frictional heat becomes darker, thus degrading the visibility of characters and images originally printed thereon.

On the other hand, as the pressure with which the pickup roller 31 presses the document decreases, the pickup roller 31 becomes unable to feed a document having a large conveyance resistance, such as a thick document or a long document.

Thus, the pressure with which the pickup roller 31 presses the document P1 is to be adjusted according to a type of the document, enabling feeding of various types of documents while preventing the stain D.

In the typical configuration, the pressure with which the pickup roller 31 presses the document is not adjusted according to a type of document that develops color upon heating, such as thermal paper. Accordingly, this configuration fails to prevent the formation of a stain D caused by frictional heat generated on a stacked document P2 at a pickup portion.

FIG. 2 is a diagram illustrating a thermal paper document after being fed by the automatic document feeder 100 in FIG. 1. The thermal paper document corresponds to the above-described stacked document P2 (a next document).

As described with reference to FIG. 1, frictional heat is generated at a position where the pickup roller 31 contacts the document P1 being fed. When the pickup roller 31 presses the document P1 with a higher pressure, the amount of frictional heat increases, and, as illustrated in FIG. 2, a stain D is formed on the stacked document P2 at a position where the pickup roller 31 contacts the stacked document P2.

An automatic document feeder 1 according to the present embodiment can reduce the contamination of a document at the sheet feeder 3 when a document that develops color upon heating, such as thermal paper, is fed.

### First Embodiment

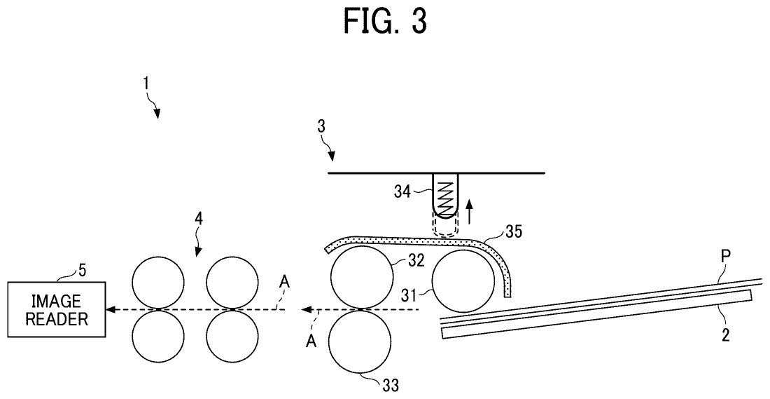

A first embodiment is described with reference to FIGS. 3 to 6.

FIG. 3 is a diagram illustrating an automatic document feeder 1 according to the first embodiment. As illustrated in FIG. 3, a document P is stacked on a document stacking portion 2. The stacked documents P are fed one by one by a sheet feeder 3 including a pickup roller 31 to a document conveyor 4 located downstream of the sheet feeder 3 in a conveyance direction A.

The sheet feeder 3 includes a pickup roller 31, a feed roller 32, and a reverse roller 33, similarly to the sheet feeder 103 illustrated in FIG. 1.

The document conveyor 4 including multiple rollers conveys the document P at a predetermined speed in the conveyance direction A. The document P conveyed by the document conveyor 4 is read by, for example, an image reader 5, and image data is acquired.

In the present embodiment, the sheet feeder 3 includes a pressure member 34 that adjusts the pressure with which the pickup roller 31 presses the document P. The pressure member 34 moves downward in FIG. 3 to contact a member 35 supporting the pickup roller 31. Thus, the pressuring force with which the pickup roller 31 presses the document P is increased. The pressure member 34 moves upward in FIG. 3 to separate from the member 35 supporting the pickup roller 31. Thus, the pressure with which the pickup roller 31 presses the document P is reduced.

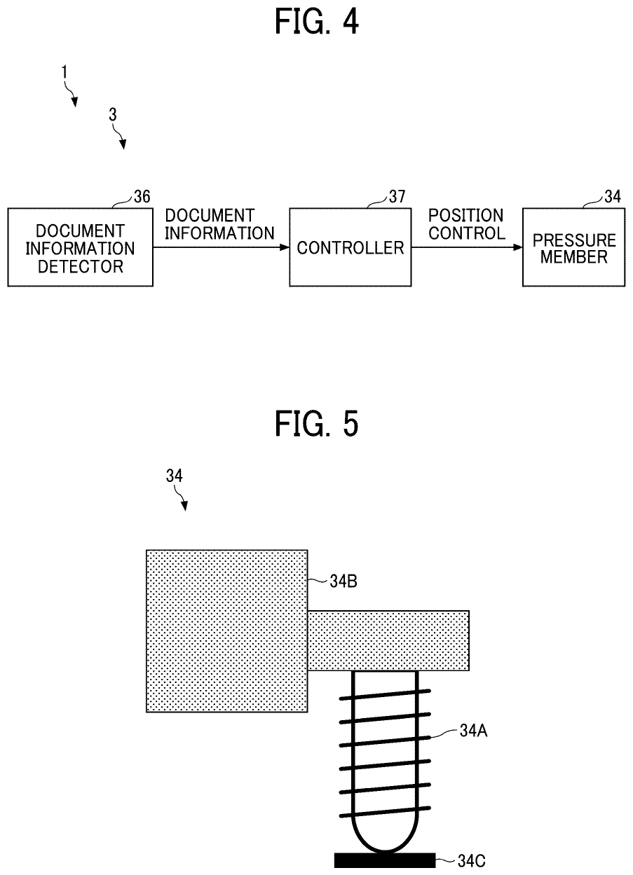

FIG. 4 is a block diagram of a sheet feeder 3 according to the first embodiment. As illustrated in FIG. 4, the sheet feeder 3 further includes a document information detector 36 and a controller 37.

The document information detector 36 detects the document type or the document size of the document P. The detected document information is transmitted to the controller 37.

The controller 37 determines whether the stacked document P is of a type that develops color upon heating (a thermally developable document), such as thermal paper, based on the document information received from the document information detector 36. The controller 37 controls a position of the pressure member 34 to change the pressure with which the pickup roller 31 presses the document, based on the determination result.

More specifically, when the controller 37 determines that the stacked document P is of a type that develops color upon heating, such as thermal paper, the controller 37 controls a position of the pressure member 34 to move the pressure member 34 away from the member 35 supporting the pickup roller 31. As a result, the pressure with which the pickup roller 31 presses the document P is reduced. This results in a reduction in frictional heat generated between the fed document P1 and the stacked document P2 (a next document; FIG. 1) at a pickup portion at which the pickup roller 31 presses the fed document P1 against the stacked document P2, preventing contamination of the stacked document P2.

When the pressure with which the pickup roller 31 presses the document P1 is reduced, conveying a document having a large conveyance resistance becomes difficult. However, since thermal paper is typically thin, the document can be conveyed without difficulty even with a small pressure applied by the pickup roller 31.

When the controller 37 determines that the stacked document P is not of a type that develops color upon heating, such as thermal paper, the controller 37 controls a position of the pressure member 34 to move the pressure member 34 into contact with the member 35 supporting the pickup roller 31. As a result, the pressure with which the pickup roller 31 presses the document P is increased. This enables a document having a large conveyance resistance to be conveyed.

The present embodiment appropriately changes the pressure with which the pickup roller 31 presses the document P according to a type of the document. This enables both prevention of contamination of a document that develops color when heated, such as thermal paper, and reliable feeding of a document having a large conveyance resistance.

FIG. 5 is a diagram illustrating a configuration of the pressure member 34 including a driving device. In FIG. 5, the pressure member 34 is a screw-type member and includes a threaded portion 34A having a screw thread cut on a rod member. The pressure member 34 includes a motor 34B at an upper portion thereof, and the motor 34B rotates the pressure member 34 to control a position of the pressure member 34. For example, the screw thread of the threaded portion 34A is in threaded engagement with a screw groove provided on the motor 34B side. In this configuration, the motor 34B is fixed at any desired position in the vertical direction. When the threaded portion 34A is rotated about an axis, the threaded portion 34A is moved in the vertical direction relative to the motor 34B via the screw thread and the screw groove, and can be positioned at any desired position in the vertical direction.

The pressure member 34 further includes a pressure plate 34C below the threaded portion 34A. The pressure plate 34C comes into contact with or is separated from the member 35 supporting the pickup roller 31 in response to vertical position control of the threaded portion 34A, so that a pressure with which the pickup roller 31 presses a document can be changed.

The configuration of the pressure member 34 is not limited to the configuration of the present embodiment illustrated in FIG. 5, and may be any electrically driven configuration that can be position-controlled by the controller 37.

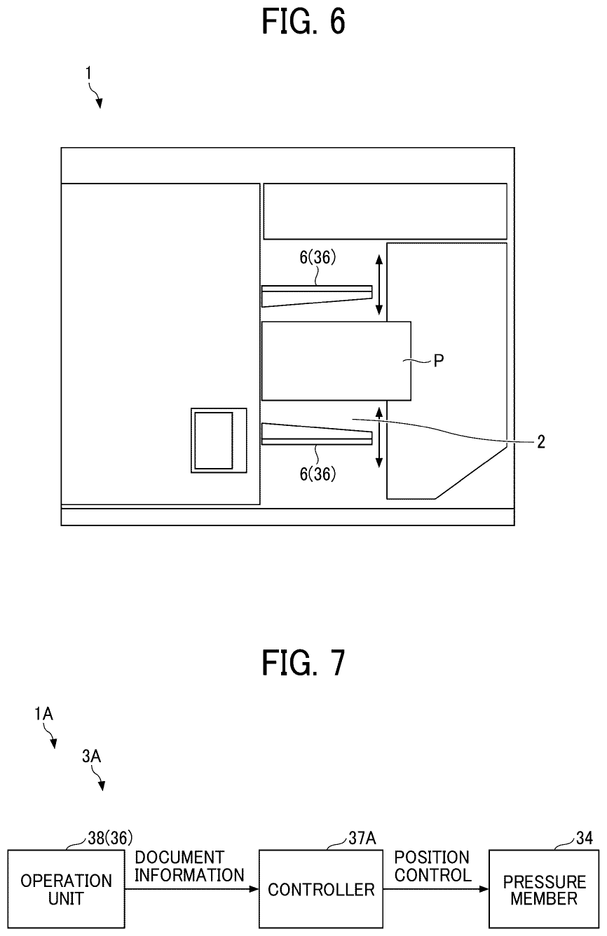

FIG. 6 is a plan view of the automatic document feeder 1 according to the first embodiment, as viewed from above. As illustrated in FIG. 6, the automatic document feeder 1 includes side fences 6 that adjust the position of the document P in the width direction to restrict the movement (skew) of the document P on the document stacker 2. The side fences 6 are movable in the width direction of the document P, adjusting the width of the document stacker 2.

In the first embodiment, the side fences 6 also serve as the document information detector 36 described above, to detect the document size based on the positions of the side fences 6.

A receipt is a typical thermal-paper document. The document width of the receipt is relatively narrow, and the document length is often longer than the document width. When such a document P is fed, the document P is likely to skew during feeding, and thus the side fences 6 are set to a width corresponding to the document width.

That is, when the controller 37 detects that the width of the side fences 6 is equal to or less than a predetermined width, the controller 37 determines that thermal paper is being fed, and controls the position of the pressure member 34 to reduce the pressure of the pickup roller 31 on the document P.

This appropriately changes the pressure with which the pickup roller 31 presses the document P according to a type of the document P. This enables both prevention of the stain D of a document P that develops color when heated, such as thermal paper, and reliable feeding of a document P having a large conveyance resistance. Further, using the side fences 6 as the document information detector 36 eliminates the need for an additional element, reducing manufacturing cost.

### Second Embodiment

FIG. 7 is a block diagram of a sheet feeder 3A according to a second embodiment.

As illustrated in FIG. 7, the sheet feeder 3A of the automatic document feeder 1A according to the second embodiment includes an operation panel 38 as the document information detector 36. As the operation panel 38, for example, an operation panel of an image forming apparatus on which the automatic document feeder 1A is mounted can be used.

The user inputs and sets various types of information relating to the document P, such as the type and thickness of the document P, via the operation panel 38. The detected document information set by the operation panel 38 is transmitted to the controller 37.

The controller 37A determines whether the stacked document P is a document that develops color in response to heating, such as thermal paper, based on the document information received from the operation panel 38. The controller 37A controls a position of the pressure member 34 to change the pressure with which the pickup roller 31 presses the document, based on the determination result.

The controller 37A may control the position of the pressure member 34 not only in two states, i.e., a contact state in which the pressure member 34 contacts the member 35 supporting the pickup roller 31 and a separated state in which the pressure member 34 is spaced apart from the member 35, but also in three or more stages by varying the pressure in the contact state. Since the type and thickness of the document P can be set in detail by the operation panel 38, the pressure can also be controlled in multiple stages according to the setting.

This allows the pressure with which the pickup roller 31 presses the document P to be more appropriately adjusted according to the type of the stacked document P. This enables both prevention of the stain D on a document P that develops color when heated, such as thermal paper, and reliable feeding of a document P having high conveyance resistance.

The information set via the operation panel 38 may include a value of the pressure with which the pickup roller 31 presses the document P. This enables the pressure to be adjusted more finely according to the type of the document P.

### Third Embodiment

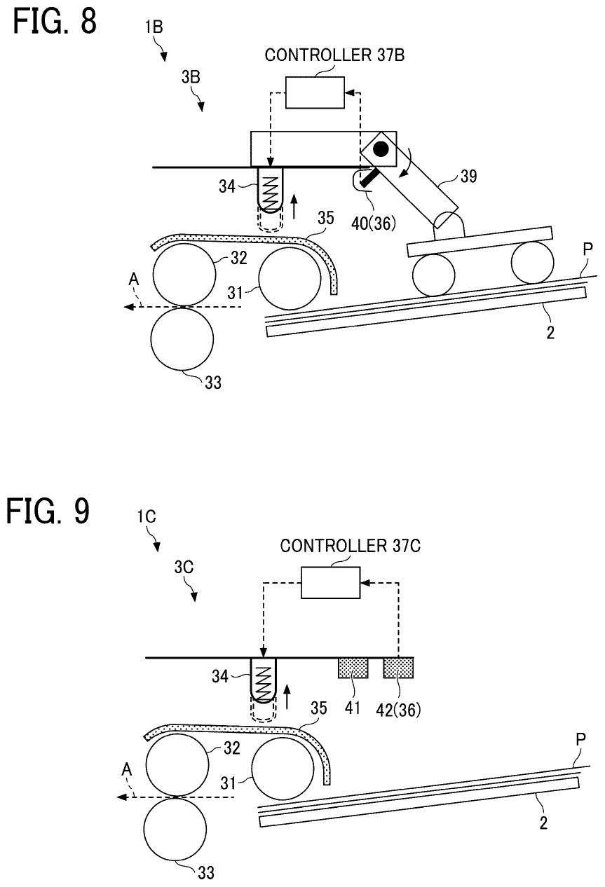

FIG. 8 is a diagram illustrating an automatic document feeder 1B according to a third embodiment.

As illustrated in FIG. 8, a sheet feeder 3B of the automatic document feeder 1B includes a pressing member 39 that restricts the skew of a document during feeding. The pressing member 39 is retractable by upward rotation. The sheet feeder 3B also includes a pressing-member detector 40 that detects that the pressing member 39 is in use.

In the third embodiment, although the document P to be conveyed is assumed to be a form such as a receipt made of thermal paper, various forms with different sizes are also used. To effectively convey the documents P, multiple forms with different sizes are collectively stacked on the document stacker 2. In this case, the side fences 6 are set to match a document having a maximum width among the stacked documents P. Accordingly, a document P having a smaller width does not come into contact with the side fences 6, and document skew may occur if the document P is fed as it is. In the third embodiment, the pressing member 39 is used to reduce or prevent such document skew.

Whether the documents P include forms such as receipts made of thermal paper can be determined based on whether the pressing member 39 is in use. That is, in the third embodiment, the pressing-member detector 40 serves as the document information detector 36.

When the pressing-member detector 40 detects that the pressing member 39 is in use, the controller 37B determines that the thermal paper is fed, and controls the position of the pressure member 34 to reduce the pressure with which the pickup roller 31 presses the document P. The present embodiment appropriately changes a pressure with which the pickup roller 31 presses the document P in accordance with a type of the document P. This enables, while reducing skew of the document P, both prevention of document stains D on a document P that develops color when heated, such as thermal paper, and reliable feeding of a document P having a large conveyance resistance.

### Fourth Embodiment

FIG. 9 is a diagram illustrating an automatic document feeder 1C according to a fourth embodiment.

As illustrated in FIG. 9, a sheet feeder 3C of the automatic document feeder 1C includes an infrared light emitter 41 that emits infrared light to a document P stacked on the document stacker 2, and an infrared light receiver 42 that receives reflection light of the infrared light emitted from the infrared light emitter 41.

Since an intensity of infrared light received by the infrared light receiver 42 varies depending on paper characteristics, the controller 37C can determine a type of the document P based on a detection result of the infrared light receiver 42. That is, in the fourth embodiment, the infrared light receiver 42 serves as the document information detector 36.

Upon receiving a signal from the infrared light receiver 42, when the signal is a predetermined signal, the controller 37C determines that thermal paper is being fed and controls a position of the pressure member 34 to reduce a pressure with which the pickup roller 31 presses the document P.

Using the infrared light receiver 42 as the document information detector 36 can eliminate a user operation for determining a type of the document P. This enables both prevention of document stains D on a document P that develops color when heated, such as thermal paper, and reliable feeding of a document P having a large conveyance resistance, without involving any operation by a user.

The controller 37A may control the position of the pressure member 34 not only in two states, i.e., a contact state in which the pressure member 34 contacts the member 35 supporting the pickup roller 31 and a separated state in which the pressure member 34 is spaced apart from the member 35, but also in three or more stages by varying the pressure in the contact state. Since the intensity of the infrared light received by the infrared light receiver 42 is a value corresponding to the paper characteristics, the controller 37C can determine whether the document P is a document P that develops color by heat, such as thermal paper, and can also distinguish documents P having different conveyance resistances. This enables adjustment of a pressure with which the pickup roller 31 presses the document P in multiple stages in accordance with the document information.

This allows the pressure with which the pickup roller 31 presses the document P to be more appropriately adjusted according to the type of the document P stacked on the document stacker 2. This enables both prevention of the stain D on a document P that develops color when heated, such as thermal paper, and reliable feeding of a document P having high conveyance resistance.

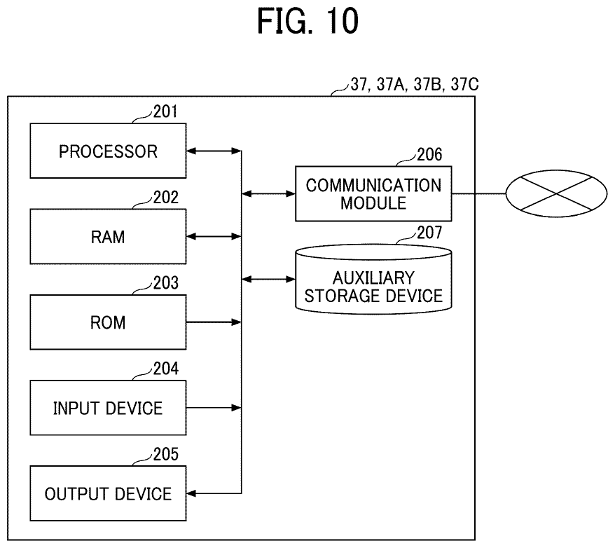

FIG. 10 is a diagram of a hardware configuration of the controllers 37, 37A, 37B, and 37C. As illustrated in FIG. 10, the controllers 37, 37A, 37B, and 37C are each physically configured as a computer system including a processor 201 including a Central Processing Unit (CPU) and a Graphics Processing Unit (GPU), a Random Access Memory (RAM) 202 and a Read Only Memory (ROM) 203 as main storage devices, an input device 204 including a keyboard and a mouse, an output device 205 including a display, a communication module 206 including a network card as a data transmitting and receiving device, and an auxiliary storage device 207 including a hard disk. The functions of the controllers 37, 37A, 37B, and 37C described above are implemented by loading predetermined computer software into the RAM 202 and executing the predetermined computer software by the processor 201. Under control of the processor 201, the predetermined computer software causes the communication module 206, the input device 204, and the output device 205 to operate, and causes data to be read from and written to the RAM 202 and the auxiliary storage device 207.

The embodiments of the present disclosure are described above with reference to specific examples. However, the present disclosure is not limited to the above-described specific examples. These specific examples to which appropriate design modifications are added by those skilled in the art are also included in the scope of the present disclosure as long as the features of the present disclosure are included. Each element included in each specific example described above and the arrangement, condition, shape, and the like thereof are not limited to those illustrated and may be changed as appropriate. The elements included in the specific examples described above may be combined as appropriate as long as there is no technical contradiction.

Aspects of the present disclosure are as follows, for example.

An automatic document feeder (1) includes a document stacker (2) to stack a document; a sheet feeder (3); a conveyor (4); a detector (36); and circuitry. The sheet feeder feeds the document from the document stacker (2). The sheet feeder (3) includes a pickup roller (31); and a pressure member (34) to apply a pressure to press the pickup roller (31) against the document on the document stacker. The conveyor (4) conveys the document fed by the sheet feeder (3) in a conveyance direction. The detector (36) detects document information including at least a type of the document on the document stacker. The circuitry is configured to determine whether the type of the document on the document stacker (2) is a thermally-developable document based on the document information detected by the detector (36); and control the pressure member (34) to change the pressure applied to the pickup roller (31) based on a determination of the type of the document on the document stacker.

### Aspect 2

In the automatic document feeder (1) according to Aspect 1, the automatic document feeder (1) further includes side fences (6) to adjust a position of the document in a width direction orthogonal to the conveyance direction on the document stacker. The detector (36) detects the document information including a size of the document on the document stacker, based on a position of the side fences.

### Aspect 3

The automatic document feeder (1) according to Aspect 1 further includes an operation panel to receive the document information. The detector (36) detects the document information received by the operation panel, as the document information of the document on the document stacker.

### Aspect 4

In the automatic document feeder (1) according to Aspect 1, the sheet feeder (3) further incudes a pressing member (39) to press an upstream side of the document in the conveyance direction on the document stacker (2) against the pickup roller. The detector (40) detects whether the pressing member (39) presses the document.

### Aspect 5

In the automatic document feeder (1) according to Aspect 1, the detector includes, at the sheet feeder (3C): an infrared light emitter (41) to emit infrared light onto the document on the document stacker (2); and an infrared light receiver (42) to receive the infrared light reflected from the document. The circuitry is further configured to determine whether the type of the document on the document stacker (2) is the thermally-developable document based on a detection of the infrared light received by the infrared light receiver (42).

### Aspect 6

In the automatic document feeder (1) according to Aspect 3 or 5, the pressure member (34) includes an actuator (34B) to change a position of the pressure member (34) to change the pressure applied to the pickup roller. The circuitry is further configured to change the position of the pressure member in three or more stages.

### Aspect 7

An automatic document feeding method includes detecting document information including at least a type of the document on a document stacker to stack the documents; determining whether the type of the document on the document stacker (2) is a thermally-developable document based on the document information detected; and controlling a pressure member (34) to change the pressure applied to a pickup roller (31) of a sheet feeder based on a determination of the type of the document on the document stacker.

The above-described embodiments are illustrative and do not limit the present invention. Thus, numerous additional modifications and variations are possible in light of the above teachings. For example, elements and/or features of different illustrative embodiments may be combined with each other and/or substituted for each other within the scope of the present invention.

The functionality of the elements disclosed herein may be implemented using circuitry or processing circuitry which includes general purpose processors, special purpose processors, integrated circuits, application-specific integrated circuits (ASICs), field-programmable gate arrays (FPGAs), and/or combinations thereof which are configured or programmed, using one or more programs stored in one or more memories, to perform the disclosed functionality. Processors are considered processing circuitry or circuitry as they include transistors and other circuitry therein. In the disclosure, the circuitry, units, or means are hardware that carry out or are programmed to perform the recited functionality. The hardware may be any hardware disclosed herein which is programmed or configured to carry out the recited functionality.

There is a memory that stores a computer program which includes computer instructions. These computer instructions provide the logic and routines that enable the hardware (e.g., processing circuitry or circuitry) to perform the method disclosed herein. This computer program can be implemented in known formats as a computer-readable storage medium, a computer program product, a memory device, a record medium such as a CD-ROM or DVD, and/or the memory of an FPGA or ASIC.

1. An automatic document feeder comprising:
a document stacker to stack a document;
a sheet feeder to feed the document from the document stacker, the sheet feeder including:
a pickup roller; and
a pressure member to apply a pressure to press the pickup roller against the document on the document stacker;
a conveyor to convey the document fed by the sheet feeder in a conveyance direction;
a detector to detect document information including at least a type of the document on the document stacker; and
circuitry configured to:
determine whether the type of the document on the document stacker is a thermally-developable document based on the document information detected by the detector; and
control the pressure member to change the pressure applied to the pickup roller based on a determination of the type of the document on the document stacker.

2. The automatic document feeder according to claim 1, further comprising side fences to adjust a position of the document in a width direction orthogonal to the conveyance direction on the document stacker wherein:
the detector detects the document information including a size of the document on the document stacker, based on a position of the side fences.

3. The automatic document feeder according to claim 1, further comprising an operation panel to receive the document information, wherein:
the detector detects the document information received by the operation panel, as the document information of the document on the document stacker.

4. The automatic document feeder according to claim 1, wherein:
the sheet feeder further incudes a pressing member to press an upstream side of the document in the conveyance direction on the document stacker against the pickup roller, and
the detector detects whether the pressing member presses the document.

5. The automatic document feeder according to claim 1, wherein:
the detector includes, at the sheet feeder:
an infrared light emitter to emit infrared light onto the document on the document stacker; and
an infrared light receiver to receive the infrared light reflected from the document, and
the circuitry is further configured to determine whether the type of the document on the document stacker is the thermally-developable document based on a detection of the infrared light received by the infrared light receiver.

6. The automatic document feeder according to claim 1, wherein:
the pressure member includes an actuator to change a position of the pressure member to change the pressure applied to the pickup roller, and
the circuitry is further configured to change the position of the pressure member in three or more stages.

7. An automatic document feeding method comprising:
detecting document information including at least a type of the document on a document stacker to stack the document;
determining whether the type of the document on the document stacker is a thermally-developable document based on the document information detected; and
controlling a pressure member to change the pressure applied to a pickup roller of a sheet feeder based on a determination of the type of the document on the document stacker.
